(19) United States
(12) Patent Application Publication
(10) Pub. No.: US 2022/0040097 A1
(43) Pub. Date: Feb. 10, 2022

(54) CHEWABLE TABLET

(71) Applicant: Genexa Inc., Atlanta, GA (US)

(72) Inventors: Max Spielberg, Beverly Hills, CA (US); David Johnson, Beverly Hills, IL (US)

(21) Appl. No.: 17/507,733

(22) Filed: Oct. 21, 2021

Related U.S. Application Data
(63) Continuation-in-part of application No. 16/014,171, filed on Jun. 21, 2018, now abandoned.

(51) Int. Cl. A61K 9/00; A61K 9/20; A61K 36/48; A61K 36/8962; A61K 36/714; A61K 35/655; A61K 36/28; A61K 36/899; A61K 36/81; A61K 36/64; A61K 36/56; A61K 36/896; A61K 36/31; A61K 36/09; A61K 33/00; A61K 36/185; A61K 36/35; A61K 36/42; A61K 36/539; A61K 38/47; A61K 35/742

(52) U.S. Cl. CPC A61K 9/0056; A61K 35/742; A61K 9/2068; A61K 9/2018; A61K 36/48; A61K 36/8962; A61K 36/714; A61K 35/655; A61K 36/28; A61K 36/899; A61K 36/81; A61K 36/64; A61K 36/56; A61K 36/896; A61K 36/31; A61K 36/09; A61K 33/00; A61K 36/185; A61K 36/35; A61K 36/42; A61K 36/539; A61K 38/47; A61K 9/2059

(57) ABSTRACT

A chewable tablet formulation includes an active material comprising an active agent in a maltodextrin base and an inactive material comprising a sugar, rice bran, carnauba wax, maltodextrin and a flavoring agent.

### RELATED APPLICATIONS

This application is a continuation-in-part of application Ser. No. 16/014,171, filed Jun. 21, 2018.

### TECHNICAL FIELD

This application relates to formulations of chewable tablets for the delivery of pharmaceuticals and nutraceuticals to patients, people or animals. More particularly, it relates to formulations of chewable tablets having improved characteristics and more natural ingredients.

### BACKGROUND

Pediatric drug development makes the availability of safe, easy-to-use dosage formulations imperative for clinical practice. Children and adults, who have difficulty swallowing conventional tablets or capsules, often benefit from chewable tablets. The advantages of chewable tablets include palatability, stability, precise dosing, portability, and ease of delivery.

Just like food and personal care products, medicine should become more natural. One of the drawbacks to oral delivery systems, however, is the situation where the drug to be administered is bitter, bad-tasting, odorous or in some manner unpleasant especially to children. The inactive ingredients can also help make a medicine look or taste better. Even conventional medications contain active ingredients that are often derived from plants, which can taste bitter. Many efforts have been made in the past to “taste mask” these compounds either through elaborate flavor and/or sweetener delivery systems, adsorption of the drug within another material or by encapsulation with a polymer, fat, carbohydrate or other like material. These taste-masking methods basically prevent the bitter tasting components of the drug from contacting the taste-buds during oral ingestion yet break down and release the active upon dissolution in the stomach.

For many years, chewable tablets have been a preferred and highly sought-after dosage form in both pharmaceutical and nutritional supplements. Like clean food, clean medicines are thought to be minimally processed and free of things like synthetic, or artificial ingredients or preservatives. Although there are many chewable tablet products available, there is a market segment that has been left unaddressed. Organic chewable pharmaceutical tablets have been allusive in the marketplace. One reason for this includes the fact that commonly known and used binders and lubricants are synthetic.

Accordingly, there is always a need for an improved chewable tablet.

### SUMMARY

This application provides a chewable tablet having (a) an active material having an ingredient mixed with a triturated maltodextrin base, and (b) a base material of suitable water-soluble sugars and/or sugar substitutes, rice bran-based excipients like Nu-Rice® and Nu-Flow®, a hydrophobic material (e.g., carnauba wax), and maltodextrin. More particularly, one aspect includes a chewable tablet having an active ingredient, dextrose, rice bran-based excipients like rice bran and rice bran extract, carnauba wax, and maltodextrin.

Another aspect includes a chewable tablet in which the hydrophobic material is selected from the group consisting of carnauba wax, beeswax, or a combination thereof.

Another aspect includes a chewable tablet in which the suitable water-soluble sugars and/or sugar substitutes are glucose, maltose, sucrose, dextrose, fructose, sorbitol, mannitol or other types of natural sweeteners. Mixtures of various sugars or sugar substitutes are also suitable.

Another aspect includes a chewable tablet that includes an active ingredient, organic dextrose, organic rice bran, organic acai berry, organic carnauba wax, organic maltodextrin. In one example, the ratio of maltodextrin to active agent can range from 1:6 to 1:13. In another example, the ratio of maltodextrin to active agent can be from 1:8-11. In yet another example, the ratio of maltodextrin to active agent can also be about 1:8.

Another aspect includes a chewable tablet in which the active ingredient is a pharmaceutical ingredient, a nutritional supplement, or combinations thereof.

Another aspect includes a method of forming a chewable tablet prepared by triturating organic maltodextrin and active agents, the maltodextrin to active agents can be in a ratio of about 1 to about 6 to 13; combining the triturated component into a mixture with dextrose, rice bran, acai berry, and organic carnauba wax; and compressing the combined mixture into a chewable tablet.

Another aspect includes a chewable tablet consisting of an active ingredient, organic dextrose, rice bran, a flavoring agent (e.g., acai berry), organic carnauba wax, organic maltodextrin. The maltodextrin to active agent ratio can be prepared by trituration prior to tableting in a ratio of about 1:6-13.

### DETAILED DESCRIPTION

Persons with knowledge of solid dosage chewable tablet formulations are familiar with the various excipients available to the formulator. These include those materials that are listed in the US Food and Drug Administration guide—Generally regarded as safe (GRAS).

Although much of the formulation development relates to chewable tablets, it has also been discovered that non-chewable tablets are also functional under the various aspects as disclosed herein. The chewable formula may be directly compressible into tablet form.

One embodiment is a chewable tablet having an active ingredient mixed with a triturated maltodextrin base, suitable water-soluble sugars, rice bran-based excipients like Nu-Rice and Nu-Flow, a hydrophobic material, and maltodextrin. This application discloses carnauba wax and tapioca dextrose in combination, as binding agents in a tablet will effectively bind materials compressed into a chewable tablet.

Specific embodiments can make use of carnauba wax that will function as both a lubricant for the chewable tablets, as well as for a tablet compression agent.

The chewable tablet includes a formulation of active ingredients mixed into maltodextrin through trituration. A tight particle size distribution may be suitable with the tablet. The process of chewable tablet production can be either direct compaction or with the aid of extra steps (e.g., dry/wet granulation). The granulation process is utilized to adhere powder particles to form larger particles or granules, which can improve the quality of tableting depending on formulas. For instance, the wet granulation process typically involves wet massing the solid ingredients of chewable formula with a liquid (e.g., water, ethanol, or isopropyl alcohol) to form wet aggregates. Then, the liquid is removed from the wet aggregates to form dry aggregates, followed by milling the dried aggregates to an appropriate size. Overwetting of granules in the wet granulation process can produce harder granules. Chewable tablets made from such wet granulations often have a gritty texture when chewed. This grittiness can be reduced by using a direct compaction manufacturing process and appropriate excipients with suitable physical-chemical properties which eliminates the wet massing and subsequent drying step.

In one embodiment, the chewable tablet can be produced by a direct compaction process. In some embodiments, the method may include preparing the excipient base formula; blending the excipient base formula with at least one active ingredient at an appropriate weight ratio to obtain a chewable formula. The chewable formula can then be compressed into a tablet of appropriate size and shape. In some embodiments, all the desired ingredients may be mixed to assure homogeneity in an appropriate blender, followed by compressing the homogeneous mixture into a tablet of appropriate size and shape. Known-tableting process (e.g., mixing and blending) can be tailored according to the demand of chewable tablet formulas.

Specific embodiments provide for the active drugs to be initially mixed with maltodextrin to use the maltodextrin as an agent to transfer the active agents into the tablet. When active agents are placed in the maltodextrin, the maltodextrin is heated to 180 degrees to assist with settling of the active agents. The active agents are then blended in the chewable tablet.

The chewable tablet can include a flavor ingredient is one sort of raw material substances which provide the senses of taste and smell to the products that utilized them. The flavor ingredients can be natural depending on their resources and processes. Natural flavor ingredients impart flavors that derive from natural substances (e.g., fruits, vegetables, and the like).

This application includes a criticality in utilizing particular ratios that have been demonstrated to impart the desired properties. It is not merely percentage composition, as chemistry remains an art where there are many observed system phenomena that are not fully understood. Thus, the ratio disclosed herein is not merely the conclusion of simple experimentation.

In one embodiment, the present invention is a chewable tablet formulation consisting of: an active ingredient, organic dextrose, organic rice bran (e.g., Nu Rice Bran®), organic acai berry, organic carnauba wax, organic maltodextrin wherein the organic maltodextrin to active ratio is prepared by trituration prior to tableting in a ratio of about 1:8-11. In one embodiment, the application provides a tablet formulation having an active ingredient, organic dextrose, organic rice bran, organic acai berry, organic carnauba wax, organic maltodextrin wherein the organic ratio of maltodextrin to active agent is prepared by trituration prior to tableting in a ratio of about 1:6-13.

In another embodiment, the active ingredient in the tablet is an active pharmaceutical ingredient, a nutritional supplement, or combinations thereof.

In another embodiment, the active ingredient in the tablet is an active pharmaceutical ingredient.

In another embodiment, the organic maltodextrin to active agent ratio is 1:8.

The present invention also includes a method of forming a chewable tablet as disclosed herein, said method comprising the steps of: preparing a trituration component of maltodextrin to active ratio in a ratio of about 1:6-13; combining said trituration component into a mixture with organic dextrose, organic rice bran or rice bran extract, organic acai berry, organic carnauba wax; and compressing said combined mixture into a chewable tablet.

In one embodiment, the method includes preparing a trituration component for a tablet of the ratio of organic ratio of maltodextrin to active agent is 1:8-11.

In one embodiment, the present invention is a tablet formulation consisting of: an active ingredient, organic dextrose, organic rice bran, organic acai berry, organic carnauba wax, organic maltodextrin wherein the organic ratio of maltodextrin to active agent is prepared by trituration prior to tableting in a ratio of about 1:6-13. In one embodiment, the present invention is a tablet formulation consisting of: an active ingredient, organic dextrose, organic Nu Rice Bran, organic Acai Berry, organic carnauba wax, organic maltodextrin wherein the organic ratio of maltodextrin to active agent is prepared by trituration prior to tableting in a ratio of about 1:8-11.

In one embodiment, the ratio of maltodextrin to active agent is prepared in a ratio of about 1:6-13. In a preferred embodiment, the ratio is about 1:8-11. The trituration includes drying the maltodextrin active combination at negative pressure for 30-60 min at 200-300 mbarr.

An “active agent” is an agent (e.g., a compound) that is permitted or approved by the U.S. Food and Drug Administration, European Medicines Agency, or any successor entity thereof, for the oral treatment of a condition or disease. Suitable pharmaceutically active agents include, but are not limited to, analgesics, anti-inflammatory agents, antipyretics, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, nicotine, and sedatives.

In another embedment, the active agent includes herbal composition effective in the management of disorders. More particularly, the invention relates to an herbal composition effective in the management of disorders stress, pain, constipation and others. The invention further relates to the use of the herbal composition in preparation of food supplements, pharmaceuticals and nutraceuticals for management of disorders related to metabolic syndrome. Also provided is a method of treating disorders comprising administering to a subject in need thereof a therapeutically effective amount of the herbal composition of the present invention.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof and pharmaceutically acceptable salts and prodrugs thereof. Examples of muscle relaxants include, but are not limited to, cyclobenzaprine and chlorzoxazone metaxalone, orphenadrine, and methocarbamol; isomers thereof; and pharmaceutically acceptable salts and prodrugs thereof. Examples of stimulants include, but are not limited to, caffeine. Examples of sedatives include, but are not limited to sleep aids such as antihistamines (e.g., diphenhydramine), eszopiclone, and zolpidem, and pharmaceutically acceptable salts and prodrugs thereof. Examples of appetite suppressants include, but are not limited to, phenylpropanolamine, phentermine, and diethylcathinone, and pharmaceutically acceptable salts and prodrugs thereof.

The percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1% , or about 1%, up to about 20% by weight, such as, e.g., from about from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition.

Examples of anesthetics (e.g., for the treatment of sore throat) include, but are not limited to dyclonine, benzocaine, and pectin and pharmaceutically acceptable salts and prodrugs thereof.

In one embodiment, the pharmaceutically active agent included within the tablet is selected from phenylephrine, dextromethorphan, pseudoephedrine, acetaminophen, cetirizine, aspirin, nicotine, ranitidine, ibuprofen, ketoprofen, loperamide, famotidine, calcium carbonate, simethicone, chlorpheniramine, methocarbomal, chlophedianol, ascorbic acid, pectin, dyclonine, benzocaine and menthol, and pharmaceutically acceptable salts and prodrugs thereof.

The active agent may also be present in the form of pharmaceutically acceptable salts, such as acidic/anionic or basic/cationic salts. Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Pharmaceutically acceptable basic/cationic salts include, and are not limited to aluminum, benzathine, calcium, chloroprocaine, choline, diethanolamine, ethylenediamine, lithium, magnesium, meglumine, potassium, procaine, sodium and zinc

The term chewing, in the context of this application, means to signify that the pulverizing or grinding is being performed by a patient's or subject's teeth, or gums. There is no intent to signify any degree of force required or generated by the teeth or gums. The requirement is that the force used to produce the pulverized granules, particles, powder and the like, is sufficient to disrupt the tablet.

The term “organic,” in the context of this application, refers to the way agricultural products are grown and processed. While the regulations vary from country to country, in the U.S., organic crops must be grown without the use of synthetic herbicides, pesticides, and fertilizers, or bioengineered genes (GMOs). The tablet formation preferably includes ingredients that must be grown without the use of synthetic herbicides, pesticides, and fertilizers, or bioengineered genes (GMOs)

### EXAMPLES

### Example 1 (Laxative)

A base material includes caramba wax, rice bran or rice bran extract, maltodextrin, dextrose and flavoring agent. The active material is a homeopathic blend of sennosides with organic maltodextrin. The base material and the active material are compressed into tablets and exhibited satisfactory friability. The active material was between 1.0 and 2.0% by weight percent, and the base material is between 98% and 99.5%. The base material contains caramba wax (2-4%), rice bran or rice bran extract (2-4%), maltodextrin (30-40%), dextrose (50-65%) and flavoring agent (less than 5%).

### Example 2 (Allergy)

A base material of caramba wax, rice bran or rice bran extract, maltodextrin, dextrose and flavoring agent (e.g., acai berry). The active material is a homeopathic blend of one or more ingredients (e.g., allium cepa, ambrosia artemisiifolia, arundo mauritanica, dulcamara, euphrasia officinalis, nux vomica, pulsatilla, ranunculus bulbosus, sabadilla, sinapis nigra, sticta pulmonaria, wyethia helenoides) with organic maltodextrin. The base material and the active material are compressed into tablets and exhibited satisfactory friability. The active material was between 1.0 and 2.0% by weight percent, and the base material is between 98% and 99.5%. The base material contains caramba wax (2-4%), rice bran or rice bran extract (2-4%), maltodextrin (30-40%), dextrose (50-65%) and flavoring agent (less than 5%).

### Example 3 (Calming)

A base material includes caramba wax, rice bran or rice bran extract, maltodextrin, dextrose and flavoring agent (e.g., vanilla). The active material is a homeopathic blend of one or more ingredients (e.g., Aconitum napellus, Calcarea phosphorica, Chamomilla, Gelsemium sempervirens, Ignatia amara, Kali carbonicum, passiflora incarnata, Valeriana officinalis) with organic maltodextrin. The base material and the active material are compressed into tablets and exhibit satisfactory friability. The active material was between 1.0 and 2.0% by weight percent, and the base material is between 98% and 99.5%. The base material contains caramba wax (2-4%), rice bran or rice bran extract (2-4%), maltodextrin (30-40%), dextrose (50-65%) and flavoring agent (less than 5%).

### Example 4 (Cold Treatment)

A base material of caramba wax, rice bran or rice bran extract, maltodextrin, dextrose and flavoring agent (e.g., acai berry). The active material is a homeopathic blend of one or more ingredients (e.g., aconitum napellus, allium cepa, bryonia alba, chamomilla, echinacea angustifolia, euphrasia officinalis, gelsemium sempervirens, ipecacuanha, pulsatilla, sambucus nigra) with organic maltodextrin. The base material and the active material are compressed into tablets and exhibited satisfactory friability. The active material was between 1.0 and 2.0% by weight percent, and base material is between 98% and 99.5%. The base material contains caramba wax (2-4%), rice bran or rice bran extract (2-4%), maltodextrin (30-40%), dextrose (50-65%) and flavoring agent (less than 5%).

### Example 5 (Sleep Treatment)

A base material includes caramba wax, rice bran or rice bran extract, maltodextrin, dextrose and flavoring agent (e.g., vanilla flavoring). The active material is a homeopathic blend of one or more ingredients (e.g., alfalfa, calcarea phosphorica, chamomilla, eschscholtzia californica, melissa officinalis, passiflora incarnata, pulsatilla, scutellaria lateriflora, valeriana officinalis) with organic maltodextrin. The base material and the active material are compressed into tablets and exhibited satisfactory friability. The active material was between 1.0 and 2.0% by weight percent, and base material is between 98% and 99.5%. The base material contains caramba wax (2-4%), rice bran or rice bran extract (2-4%), maltodextrin (30-40%), dextrose (50-65%) and flavoring agent (less than 5%).

### Example 6 (Antacid)

A base material includes caramba wax, rice bran or rice bran extract, maltodextrin, dextrose and flavoring agent. The active material is a homeopathic blend of calcium carbonate with organic maltodextrin. The base material and the active material are compressed into tablets and exhibited satisfactory friability. The active material was between 1.0 and 2.0% by weight percent, and base material is between 98% and 99.5%. The base material contains caramba wax (2-4%), rice bran or rice bran extract (2-4%), maltodextrin (30-40%), dextrose (50-65%) and flavoring agent (less than 5%).

### Example 7

Organic Dextrose, Microcrystalline Cellulose, Lactose, Organic Acai Berry Flavor

Besides the Organic Sugar (Dextrose), there is not much choice for lubrication (Silicon Dioxide, Magnesium Stearate) due to the need for the product to be 100% certified organic/natural ingredients, and therefore there are sticking problems of the tablets to the punches and the edges of the tablets are also not even. Tablets exhibit variable compression characteristics.

### Example 8

Organic Dextrose, Microcrystalline Cellulose, Organic Nu Rice Bran, Organic Carnauba Wax, Organic Acai Berry, Organic Rice Hull. Tablets exhibited satisfactory compression and friability. Organic Rice Hull was added but it affected the taste & color. Adding the Organic Carnauba Wax did not have much of an effect.

### Example 9

Organic Dextrose, Microcrystalline Cellulose, Organic Nu Rice Bran, Organic Carnauba Wax, Organic Acai Berry, and Organic Maltodextrin. Organic maltodextrin was used instead of the rice hull we have been using. Tablets exhibited satisfactory compression and friability.

### Example 10

Vitamin D, organic dextrose, organic rice bran, organic acai berry, organic carnauba wax, organic maltodextrin, and flavoring was used in the tablets. Tablets exhibited satisfactory compression and friability. Ratio of maltodextrin to vitamin D was about 4.5.

### Example 11

Lactase, bacillus coagulans, Organic Nu Rice Bran, Organic Acai Berry, Organic Carnauba Wax, Organic Maltodextrin was used in the tablets. Tablets exhibited satisfactory compression and friability.

### Example 12

Senna herbs and senna extract, Nu Rice Bran, flavoring agents, Organic Carnauba Wax, and organic maltodextrin was used in the tablets. Tablets exhibited satisfactory compression and friability.

### Example 13 (Cramps)

A base material includes caramba wax, rice bran or rice bran extract, maltodextrin, dextrose and flavoring agent (e.g., acai berry). The active material is a homeopathic blend of one or more ingredients (angustura vera, berberis vulgaris, chamomilla, colchicum autumnale, colocynthis, ignatia amara, jatropha curcas, lycopodium clavatum, nux moshchata, rhus toxicodendron, secale cornutum, veratrum album) with organic maltodextrin. The base material and the active material are compressed into tablets and exhibited satisfactory friability. The active material was between 1.0 and 2.0% by weight percent, and base material is between 98% and 99.5%. The base material contains caramba wax (2-4%), rice bran or rice bran extract (2-4%), maltodextrin (30-40%), dextrose (50-65%) and flavoring agent (less than 5%).

### Example 14 (Cramps)

A base material includes caramba wax, rice bran or rice bran extract, maltodextrin, dextrose and flavoring agent (e.g., acai berry). The active material is a homeopathic blend of one or more ingredients (angustura vera, berberis vulgaris, chamomilla, colchicum autumnale, colocynthis, ignatia amara, jatropha curcas, lycopodium clavatum, nux moshchata, rhus toxicodendron, secale cornutum, veratrum album) with organic maltodextrin. The base material and the active material are compressed into tablets and exhibited satisfactory friability. The active material was between 1.0 and 2.0% by weight percent, and base material is between 98% and 99.5%. The base material contains caramba wax (2-4%), rice bran or rice bran extract (2-4%), maltodextrin (30-40%), dextrose (50-65%) and flavoring agent (less than 5%).

### Example 15 (Motion Sickness)

A base material includes caramba wax, rice bran or rice bran extract, maltodextrin, dextrose and flavoring agent (e.g., acai berry). The active material was a homeopathic blend of one or more ingredients (bryonia alba, cinnamonum, cocculus indica, colchicum autumnale, iris versicolor, kali carbonicum, nux moschata, staphysagria, tabacum, zingiber officinale) with organic maltodextrin. The base material and the active material are compressed into tablets and exhibited satisfactory friability. The active material was between 1.0 and 2.0% by weight percent, and base material is between 98% and 99.5%. The base material contained caramba wax (2-4%), rice bran or rice bran extract (2-4%), maltodextrin (30-40%), dextrose (50-65%) and flavoring agent (less than 5%).

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A chewable tablet formulation comprising: (a) an active material comprising an active agent in a maltodextrin base and (b) an inactive material comprising a sugar, rice bran, carnauba wax, maltodextrin and a flavoring agent.

2. The tablet of claim 1, wherein the active ingredient is a nutritional supplement.

3. The tablet of claim 1, wherein the active ingredient is an herbal ingredient.

4. The tablet of claim 1, wherein the organic ratio of maltodextrin to active agent is prepared by trituration prior to tableting in a ratio of about 1:6-13.

5. The tablet of claim 1, wherein the active agent is an active pharmaceutical ingredient.

6. The tablet of claim 1, wherein the maltodextrin to active ingredient is in ratio of between 1 to 6 and 1 to 13.

7. The tablet of claim 1, wherein the maltodextrin to active ingredient is in ratio of between 1 to 8 and 1 to 11.

8. The tablet of claim 1, wherein the active ingredient and the maltodextrin base are triturated.

9. The tablet of claim 6, wherein the caramba wax is between 2 and 4% by weight, the rice bran or rice bran extract is between 2 and 4% by weight, maltodextrin is between 30 and 40% by weight, dextrose is 50 and 65% by weight, and the flavoring agent is less than 5% by weight.

10. The tablet of claim 6, wherein the caramba wax is between 2 and 3% by weight, the rice bran or rice bran extract is between 2 and 4% by weight, maltodextrin is between 30 and 40% by weight, dextrose is 50 and 65% by weight, and the flavoring agent is less than 5% by weight.

11. The tablet of claim 1, wherein the active ingredient is herb.

12. A method of forming a chewable tablet, comprising the steps of:
a. preparing a trituration component of maltodextrin and an active ratio in a ratio of about 1:6-13;
b. combining said trituration component into a mixture; and
c. compressing said combined mixture into a chewable tablet.

13. The method of claim 8, wherein the mixture has a sugar, a hydrophobic material, and a rice bran.

14. The method of claim 8, wherein the mixture is a flavoring agent.

15. The method of claim 8, wherein the ratio of the maltodextrin to the active ratio is 1 to 8.

16. A chewable tablet formulation comprising: (a) an active material comprising an herbal agent in a maltodextrin base and (b) an inactive material comprising a sugar, rice bran, carnauba wax, maltodextrin and a flavoring agent,
wherein the organic maltodextrin to active ratio is prepared by trituration prior to tableting in a ratio of about 1:6-13.

17. The tablet formulation of claim 12 wherein said active ingredient in the tablet is an active pharmaceutical ingredient, a nutritional supplement, or combinations thereof.

18. The tablet formulation of claim 7 wherein said organic maltodextrin to active ratio is 1:8-11.

19. The tablet of claim 16, wherein the caramba wax is between 2 and 4% by weight, the rice bran or rice bran extract is between 2 and 4% by weight, maltodextrin is between 30 and 40% by weight, dextrose is 50 and 65% by weight, and the flavoring agent is less than 5% by weight.

20. A chewable tablet formulation comprising essentially: (a) an active material comprising an active agent in a maltodextrin base and (b) an inactive material comprising a sugar, rice bran, carnauba wax, maltodextrin and a flavoring agent.
